(19) United States
(12) Patent Application Publication
(10) Pub. No.: US 2001/0007154 A1
(43) Pub. Date: Jul. 5, 2001

(54) MATERIALS AND METHODS FOR HYBRID SEED PRODUCTION

(75) Inventors: DIANE BURGESS, BERKELEY, CA (US); NEAL GUTTERSON, OAKLAND, CA (US)

Correspondence Address: TOWNSEND AND TOWNSEND AND CREW, TWO EMBARCADERO CENTER, EIGHTH FLOOR, SAN FRANCISCO, CA, 94111-3834, US

(73) Assignee: DNA PLANT TECHNOLOGY CORPORATION

(21) Appl. No.: 09/186,775

(22) Filed: Nov. 6, 1998

Related U.S. Application Data
(63) Continuation-in-part of application No. 09/012,895, filed on Jan. 23, 1998.
(60) Provisional application No. 60/065,989, filed on Nov. 14, 1997.
(60) Provisional application No. 60/036,483, filed on Jan. 24, 1997.

(51) Int. Cl. A01H 5/00

(52) U.S. Cl. 800/260

(57) ABSTRACT

The present invention is directed to methods for producing plants containing alternate expression cassettes at a single locus in the plant genome. The two expression cassettes encode polypeptides which, when present in the same cell, are lethal to the cell. In preferred embodiments, the plant cell is an anther cell and the plant is male sterile.

### CROSS-REFERENCE TO RELATED APPLICATIONS

This is a continuation-in-part of U.S. Ser. No. 60/036,483, filed Jan. 24, 1997, U.S. Ser. No. 09/012,895, filed Jan. 23, 1998 and U.S. Ser. No. 60/065,989, filed Nov. 14, 1997, which are incorporated herein by reference.

### FIELD OF THE INVENTION

The present invention relates generally to methods for preventing the growth of specific cells in plants. In particular, the invention provides male sterile plants useful in the production of hybrid seed.

### BACKGROUND OF THE INVENTION

One objective of plant genetic engineering is to create novel traits through targeted expression of an introduced gene. One use of targeted gene expression is the elimination of specific plant cells through the production of proteins that are lethal to the cell. In order to eliminate only a specific set of cells, it is necessary that expression of a potentially lethal function be controlled precisely such that the cell-lethal function is expressed only in the cells targeted for elimination and in no others.

For instance, by targeting expression to cells in the tapetum, male sterile plants can be produced. The production of male sterile plants is particularly useful in producing F1 hybrids. F1 hybrid plants are used extensively in most areas of agriculture because of their improved traits, such as increased yield, disease or low temperature resistance. F1 hybrids are often produced by a manual process of emasculation of the intended female of the cross, to prevent self pollination, followed by application of pollen taken from the male of the cross to the stigma of the female of the cross. The production of such hybrids is labor intensive, which contributes greatly to the increased cost of hybrid seed.

Several different approaches have now been attempted to use cell lethality systems in the production of male sterile lines by targeting expression of lethal protein to the tapetum. For example, WO 96/26283, describes the production of male sterility using the tapetal specific promoter TA29 from tobacco to program expression of the ribonuclease, barnase. U.S. Pat. No. 5,409,823 discloses use of transactivators to control expression of gene products which disrupt formation of pollen. Since it is often desirable that the male fertility be restored in either the male sterile line or the F1 hybrid, attempts have been made to produce plants in which male sterility can be made conditional. Examples of this approach include WO 93/25695 and WO 97/13401.

Although progress has been made, the prior art lacks efficient methods by which selected plant cells can be eliminated, but that also provide means by which cell lethality can be controlled so that cell function can be restored, if desired. The present invention provides these and other advantages.

### SUMMARY OF THE INVENTION

The present invention provides plants containing a plant cell comprising a first and a second expression cassette located at the same locus on each of two homologous chromosomes. One expression cassette comprises a first plant promoter operably linked to a first polynucleotide sequence encoding a first polypeptide. A recombinase site (e.g., a lox site) is present between the first promoter and the first polynucleotide sequence. A second expression cassette comprises the first plant promoter inoperably linked to the first polynucleotide sequence, wherein an intervening expression cassette is flanked by two recombinase sites and situated between the first promoter and the first polynucleotide sequence of the second expression cassette. The intervening expression cassette comprises a second plant promoter operably linked to a second polynucleotide sequence encoding a second polypeptide. The presence of the first and second polypeptides in a cell is lethal to the cell.

The first and second polypeptide can be selected from a number of proteins, which when present together are lethal to a cell. For instance, one polypeptide can be a transactivator protein which activates expression of the other expression cassette which encodes a polypeptide which is lethal to plant cells (e.g., a ribonuclease). Alternatively, the polypeptides can be an avirulence gene product derived from a plant pathogen and a plant resistance gene product associated with the avirulence gene (e.g., AVR9 and CF9).

The promoters in the two expression cassettes preferably provide tissue specific expression of one or both of the polypeptides. In some embodiments, the target cells are tapetal cells.

Methods of preparing plants of the invention are also provided. For instance, the plants can be prepared by introducing into a plant the expression cassettes described above.

### BRIEF DESCRIPTION OF THE FIGURES

FIG. 1 diagrams the production of allelic variants of the invention using the cre/lox recombinase system and a transactivator construct. ALS=acetolactate synthase.

FIG. 2 shows the production of allelic variants of the invention using the cre/lox system and AVR9/CF9. ALS=acetolactate synthase.

### DETAILED DESCRIPTION OF THE INVENTION

The present invention provides methods for inhibiting the growth or killing of specified plant cells. More specifically, the present invention relates to plant cells comprising at least two expression cassettes operably linked to polynucleotides which when expressed in the same cell are lethal to the cell. As explained below, present invention provides novel methods for producing plants in which the expression cassettes occupy the same locus on chromosome homologs. Thus, targeted cells in plants homozygous at the locus (i.e., have the same expression cassette on each homolog) are not eliminated. Targeted cells in plants heterozygous at the locus (i.e., have a different expression cassette on each homolog) are eliminated.

Methods of the present invention provide means to maintain inbred lines in a hybrid system in which there is no inhibitory or lethal activity expressed in either inbred line. However, crossing these inbred lines yields a hybrid having the inhibitory or lethal phenotype. The resulting invention has utility, for example, in creating and maintaining male sterile and female sterile plants.

Definitions

Units, prefixes, and symbols can be denoted in their SI accepted form. Numeric ranges are inclusive of the numbers defining the range. The headings provided herein are not limitations of the various aspects or embodiments of the invention which can be had by reference to the specification as a whole. Accordingly, the terms defined immediately below are more fully defined by reference to the specification in its entirety.

As used herein, the term “plant” includes reference to whole plants, plant organs (e.g., leaves, stems, roots, etc.), seeds and plant cells and progeny of same. The class of plants which can be used in the methods of the invention is generally as broad as the class of higher plants amenable to transformation techniques, including both monocotyledonous and dicotyledonous plants. It includes plants of a variety of ploidy levels, including polyploid, diploid and haploid.

As used herein “operably linked” includes reference to a functional linkage between a promoter and a second sequence, wherein the promoter sequence initiates and mediates transcription of the DNA sequence corresponding to the RNA sequence which is typically transcribed into a polypeptide. Generally, operably linked means that the nucleic acid sequences being linked are contiguous and, where necessary to join two protein coding regions, contiguous and in the same reading frame.

As used herein, a “expression cassette” is a nucleic acid construct, generated recombinantly or synthetically, with a series of specified nucleic acid elements which permit transcription of a particular nucleic acid in a target cell. The expression cassette can be incorporated into a plasmid, chromosome, mitochondrial DNA, plastid DNA, virus, or nucleic acid fragment. Typically, the expression cassette portion of the expression vector includes, among other sequences, a nucleic acid to be transcribed, and a promoter.

As used herein, “lethal” or “impairs cellular function” includes reference to polynucleotide(s) or polypeptide(s) that are cytotoxic to an extent that kills cells or inhibits cell division or differentiation. Thus, “lethal” or “impairs cellular function” includes reference either to 1) the disruption of a cell through perturbation of some function of the cell or by degradation of a component of the cell, or 2) to the prevention of continued growth of a cell through perturbation of some function of the cell or degradation of some component of the cell. By way of example, but not limitation, typical cellular functions in the context of the instant invention are protein synthesis, RNA synthesis, protein maintenance of osmotic competence, lipid synthesis, DNA synthesis. Typical cellular components subject to degradation in the context of the instant invention are proteins, carbohydrates, membranes, deoxyribonucleic acids, ribonucleic acids.

The terms “chromosome homolog” or “homologous chromosome” refers to two or more chromosomes that can pair during meiosis. Each homologue is a duplicate of a chromosome contributed by the male or female parent. Homologous chromosomes contain the same linear sequence of genes, each gene (or allele) is present at the same locus on each homolog.

Two polynucleotide sequences (e.g., two expression cassettes of the invention) are said to be at the “same locus” if the two sequences are genetically mapped to the same locus as determined, for instance, by frequency of crossover events between the two loci.

As used herein, “heterologous” is a nucleic acid that originates from a foreign species, or, if from the same species, is substantially modified from its original form. In the present disclosure, a promoter operably linked to a heterologous structural gene is from a species different from that from which the structural gene was derived, or, if from the same species, one or both are substantially modified from their original form, for example, the promoter can be linked to a structural gene from the same species, but not the one to which it is normally operably linked. Thus, a “heterologous expression cassette” is one that comprises at least one element not endogenous to the species or subspecies in which it is introduced.

As used herein, “polynucleotide” includes reference to both double stranded and single stranded DNA or RNA. The terms also refer to synthetically or recombinantly derived sequences essentially free of non-nucleic acid contamination. A polynucleotide can be a gene subsequence or a full length gene (cDNA or genomic).

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein, “functional” includes reference to an activity sufficient to produce a desired effect. Thus, for example, a promoter functional in a specified cell will drive expression to the desired levels. A “functional polypeptide” will have the activity to achieve a desired result, such as cell inhibition or death. A “functional expression cassette” is one in which a promoter is operably linked to a second sequence encoding a desired polypeptide and/or RNA sequence.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that initiate transcription only in certain organs or tissues, such as leaves, roots, fruit, seeds, tapetal tissue, anthers, stigmas, or flowers. Such promoters are referred to as “tissue specific”. A “cell type” specific promoter primarily drives expression in certain cell types in one or more organs, for example, vascular cells in roots or leaves. An “inducible” promoter is a promoter in which the rate of transcription is increased in the presence of a transactivator protein or other inducing signal. Examples of transactivator proteins include the repressor/activator fusion proteins described below. Inducible promoters can be activated by environmental conditions such as anaerobic conditions or the presence of light. Tissue specific, cell type specific, and inducible promoters constitute the class of “non-constitutive” promoters. A “constitutive” promoter is a promoter which is active under most environmental conditions and in most tissues.

Plant Compositions and Methods

The present invention has use over a broad range of types of plants, including species from the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum and Datura.

Plant cells of the present invention comprise two or more expression cassettes. When the promoters of both expression cassettes are functional in a cell, the cell is eliminated. The two expression cassettes are present at identical loci locus in a homologous pair. The first cassette may be derived from the second cassette using a recombinase system. An example of a recombinase useful in the invention is the cre/lox recombinase system. Bayley et al., Plant Molecular Biology, 18:353-361 (1992) and U.S. Pat. No. 4,959,317. Other recombinase systems useful in the invention include the Saccharomyces cerevisiae FLP/FRT, lambda att/Int, R recombinase of Zygosaccharomyces rouxii, and Mu Gin recombinase. Alternatively, one of the expression cassettes can include a transposable element such as Ds from maize which could be excised by crossing to a line carrying a transposase.

In one embodiment of the invention, an expression cassette which can subsequently be manipulated via a recombinase to remove an intervening subsequence of that cassette is introduced into a plant. The intervening subsequence is positioned between recombinase sites in the same orientation, such that the promoter of the expression cassette is inoperably linked to a coding sequence. In the presence of the appropriate recombinase enzyme the intervening subsequence is excised from the expression cassette such that the promoter is now operably linked to the corresponding coding sequence. Alternatively, the recombinase can be used to insert an intervening subsequence into a recombinase site in an expression cassette previously introduced into the plant. In the presence of the appropriate recombinase enzyme the intervening subsequence is inserted into the expression cassette such that the promoter is now inoperably linked to the corresponding coding sequence. Using these methods alternate functional cassettes can be introduced to a particular locus in the genome. One of the cassettes will be a nonfunctional expression cassette (with the intervening sequence) and the other of the cassettes will be a functional expression cassette (without the intervening sequence).

In the invention, the intervening sequence also includes an intervening expression cassette encoding a second polypeptide. The presence of polypeptides from both expression cassettes is lethal to the cell.

In one method of making plants of the invention, an initial plant line is created which contains a non-functional expression cassette interrupted by an intervening expression cassette flanked by recombinase sites in the same orientation (e.g., lox sites). This initial line is then crossed with a second plant containing the appropriate recombinase (e.g., cre). Plants in the F1 generation will contain a functional expression. F1 plants containing a functional cassette are then selfed according to standard techniques to produce a homozygous line containing a functional expression cassette encoding the first polypeptide. The original transformant is also selfed to produce a second line in which the first expression cassette remains non-functional. The two lines are then crossed to produce plants heterozygous at the locus. Cells in which both expression cassettes are functional will be eliminated.

In another method, an initial plant line is created which contains a functional expression cassette containing a recombinase site (e.g., a lox site) between the promoter and the structural gene. To create a second plant line, a plasmid containing a an intervening expression cassette, and a second recombinase site (e.g., a lox site) is introduced into tissue or cells (e.g., protoplasts) from the initial plant along with a second plasmid encoding the appropriate recombinase (e.g., cre). The second plant line is then regenerated from the transformed tissue or cells. A recombination event between the plasmid DNA continuing the intervening expression cassette[←Is the preceding phrase of 5 words meant to be inserted here as well??] and the first expression cassette will result in insertion of the plasmid DNA continuing the intervening expression cassette into the first expression cassette, thereby rendering the first expression cassette non-functional and thereby yielding the second expression cassette. The two lines are then crossed to produce plants containing the two cassettes at the same locus on each of the two homologous chromosomes heterozygous at the locus. Cells in which both expression cassettes are functional will be eliminated.

In these embodiments, it is desirable to use combinations of mutant lox sites to increase the relative efficiency of the insertion event or excision. Mutant lox sites (e.g., lox₆₆ and lox₇₇) are described by Albert et al. Plant J. 6:649-659 (1995)), The expression cassettes of the present invention are DNA or RNA constructs which can be cloned and/or synthesized by any number of standard techniques. An expression cassette will typically comprise transcriptional and translational initiation regulatory sequences which will direct the transcription of the polynucleotide encoding a non-lethal polypeptide in the intended tissues of the transformed plant. Such nucleic acid constructs may be introduced into the genome of the desired plant host by a variety of conventional techniques. Techniques for transforming a wide variety of higher plant species are well known and described in the technical and scientific literature. See, for example, Weising et al. Ann. Rev. Genet. 22:421-477 (1988).

For example, the DNA or RNA nucleic acid construct may be introduced directly into the genomic DNA of the plant cell using techniques such as electroporation and microinjection of plant cell protoplasts, or the nucleic acid constructs can be introduced directly to plant tissue using ballistic methods, such as DNA particle bombardment. Alternatively, the nucleic acid constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, for example Horsch et al. Science, 233:496-498 (1984), and Fraley et al. Proc. Natl. Acad. Sci. USA 80:4803 (1983).

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

The expression cassettes of the present invention can comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulforon or Basta.

Promoters

The promoters employed in the expression cassettes of the present invention can be chosen to function in particular tissue types. A very wide range of promoters can be used with the multi-component system of the present invention. Methods for identifying promoters with a particular expression pattern, in terms of, e.g., tissue type, cell type, stage of development, and/or environmental conditions, are well known in the art. A typical step in promoter isolation methods is identification of gene products that are expressed with some degree of specificity in the target tissue. Amongst the range of methodologies are: differential hybridization to cDNA libraries; subtractive hybridization; differential display; differential 2-D gel electrophoresis; isolation of proteins known to be expressed with some specificity in the target tissue. Such methods are well known to those of skill in the art.

In the process of isolating promoters expressed under particular environmental conditions or stresses, or in specific tissues, or at particular developmental stages, a number of genes are identified that are expressed under the desired circumstances, in the desired tissue, or at the desired stage. Further analysis will reveal expression of each particular gene in one or more other tissues of the plant.

Once promoter and/or gene sequences are known, a region of suitable size is selected from the genomic DNA that is 5′ to the transcriptional start, or the translational start site, and such sequences are then linked to a partial coding sequence as described above. If the transcriptional start site is used as the point of fusion, any of a number of possible 5′ untranslated regions can be used in between the transcriptional start site and the partial coding sequence. If the translational start site at the 3′ end of the specific promoter is used, then it is linked directly to the methionine start codon of a partial coding sequence.

To identify the promoters, the 5′ portions of the clones described here are analyzed for sequences characteristic of promoter sequences. For instance, promoter sequence elements include the TATA box consensus sequence (TATAAT), which is usually 20 to 30 base pairs upstream of the transcription start site. In plants, further upstream from the TATA box, at positions −80 to −100, there is typically a promoter element with a series of adenines surrounding the trinucleotide G (or T) N G. J. Messing et al., in Genetic Engineering in Plants, pp. 221-227 (Kosage, Meredith and Hollaender, eds. 1983). If proper polypeptide expression is desired, a polyadenylation region should be included. The polyadenylation region can be derived from the 3′ end of a natural gene, from a variety of other plant genes, or from T-DNA.

Modification of the promoter characterized as described herein can be done using any of a number of methods well known in the art. For example, specific enhancer sequences can be added to the promoter to increase the expression level or to modify the expression pattern. Further, an intron sequence can be added to the 5′ untranslated region or the coding sequence of the partial coding sequence to increase the amount of the mature message that accumulates in the cytosol.

As noted above, one use of the present invention is the production of male sterile plants. Thus, promoter combinations which lead to tissue-specific expression in anther tissues (e.g., tapetal tissues) are useful in the invention. Targeted expression of the desired gene products can be achieved in a number of ways. For instance, a combination of promoters whose expression overlaps only in anther tissue can be used. Alternatively, one expression cassette can include a constitutive promoter, while the second includes a tissue-specific promoter. In other embodiments, two tissue-specific promoters are used. Tapetal-specific promoters are particularly useful in the invention. Examples of such promoters include TA29 from tobacco (Mariani et al., Nature, 347:737-41, (1990)), 127a, 108, 92b, 101B, and 5B from tomato (Chen and Smith, Plant Physiol., 101:1413 (1993), Smith et al. Mol. Gen. Genet. 222:9-16 (1990) Aguirre and Smith, Plant Mol. Biol., 23:477-87, (1993)), tap1 from Antirrhimum majus (Nacken et al. Mol. Gen. Genet. 229:129-136 (1991), and A6 and A9 from Brassica (Paul et al., Plant Mol. Biol., 19:611-22, (1992), Hird et al. Plant Journal 4:1023-1033 (1993)). Anther-specific promoters could also be used such as ones isolated by Twell et al. (Mol. Gen. Genet., 217:240-45, (1991)). Seed coat specific promoters, such as the pT218 promoter (Fobert et al., The Plant Journal, 6:567-77, 1994) or the pWM403 promoter could also be used in the present invention. Tissue-specific promoters for a range of different tissues have been identified, including roots, sepals, petals, and vascular elements. In addition, promoters induced upon pathogen infection have been identified, such as the prp-1 promoter (Strittmatter et al., Bio/Technology, 13, 1085-90, (1995)). Promoters induced in specialized nematode feeding structures have been identified (disclosed in patent applications WO 92/21757, WO 93/10251, WO 93/18170, WO 94/10320, WO 94/17194).

In some embodiments the promoter in one of the expression cassettes is a promoter inducible by the gene product of the second expression cassette. In these embodiments, the gene product of the inducible expression cassette is, by itself, lethal to the plant cell. Tapetal-specific expression of genes such as ribonucleases (e.g., Barnase), or premature expression of β-1,3 glucanases in the tapetum, have been shown to produce male sterility. Examples of other lethal polypeptides and nucleic acids are set forth below. In these embodiments, the second expression cassettes may encode a repressor/activator fusion protein. These proteins use activator domains fused to prokaryotic repressor domains thus turning them into transcriptional activators (see, e.g., Brent et al. Cell 43:729-736 (1985) and Labow et al. Mol. Cell. Biol. 10:3343-3356 (1990). The repressor domains recognize specific sequences in the target promoter while the activator domains provide transcriptional activator function. An exemplary fusion protein for this purpose is a fusion between the Tn10 encoded tet repressor and the activation domain of the Herpes simplex protein VP16 (Weinmann et al. The Plant Journal 5:559 (1994). In these embodiments, the promoter will be a tet artificial promoter comprising at least one tet operator and a TATA-box (as described by Weinman et al.).

Other operator recognition systems that can be used include lacR/O, GAL4, and 434R/O. [The TnpA binding protein from maize Spm, when fused to an activator domain such as VP16, can be used to transactivate the Spm promoter (Schläppi et al., Plant Mol. Biol. 32:717-725 (1996)).] Other activator domains which can be employed in the present invention include the acid domains from Vp1, ABI3, PvAlf, HAP4, and GCN4. Non-acidic activator domains can also be used, such as proline-rich domains, serine/threonine-rich domains, and glutamine-rich domains. [Transactivator polypeptides are not limited to repressor/activator fusions, but include naturally occurring transactivator polypeptides such as the transcriptional activator polypeptides expressed by geminiviruses. These include the AL2 gene product from Tomato Golden Mosaic Virus (TGMV) which transactivates expression of the TGMV coat protein and BR1 movement protein genes and BR1 movement protein genes (Sunter et al., Virology 232:269-280 (1997)), and the AC2 gene product of the African Cassava Mosaic Virus (ACMV) which transactivates expression of the ACMV coat protein.]

Lethal Effects

Except in embodiments in which the gene product of one expression cassette induces expression of the second expression cassette, the product of each expression cassette of the present invention individually is not lethal, by itself. It takes the combination of all transcripts (typically translated into polypeptides) from the individual expression cassettes to result in the desired phenotype. For example, lethal or inhibitory transcripts can provide sense or antisense suppression, or lethal or inhibitory transcripts can be translated into a prozyme which is activated upon processing by a specific protease which is the product of the other expression cassette. Prozymes can be artificially created by linking a desired “pro” region to an active enzyme through a linker containing recognition sequences of a desired protease. Examples of proteases useful in the invention include proteases from polyviruses such as the NIa proteinase from tobacco etch virus or tobacco vein mottling virus (see, e.g., Parks and Dougherty Virology 182:17-27 (1991)).

Polypeptides

Examples of polypeptides include avirulence/resistance gene combinations which lead to a hypersensitive response and cell death. Examples of this system are the AVR elicitor polypeptides from Cladosporium fulvum and the corresponding resistance genes, Cf from Lycopersicon [some Cf genes originated in other Lycopersicon species from which they were then introgressed[??] into L. esculentum] (e.g., Cf2/Avr2, Cf4/Avr4, Cf5/Avr5, and Cf9/Avr9, see, Jones et al. Science 266:789-793 (1994) and Hammond-Kosack and Jones Plant Cell 8:1773-1791 (1996)). A preferred combination is Cf9/Avr9. A hypersensitive response is elicited in cells expressing both Avr9 and Cf9 and results in cell death. In preferred embodiments, the AVR peptide is linked to a sequence targeting it to the apoplast (see, e.g., Hammond-Kosack et al. Proc. Natl. Acad. Sci. USA 91:10445-10449). Other avirulence/resistance gene combinations include the tomato Pto gene and the Pseudomonas syringae avrPto avirulence gene (Martin et al. Science 262:1432 (1993), the RPS2 gene of Arabidopsis thaliana confers resistance to P. syringae that express the avrRpt2 avirulence gene (Bent et al. Science 265:1856-1860 (1994)), and the tobacco N gene and TMV replicase (Padgett et al. Molecular Plant Microbe Interactions 10709-715 (1997)).

Polypeptides of the present invention can also be derived from overlapping or non-overlapping subsequences of a single functional protein which provides for the desired phenotype when co-expressed in a cell. Additionally polypeptides of the present invention can consist of separate monomers of a lethal dimeric protein. In some embodiments the polypeptides will be a prozyme and a protease which processes the prozyme and renders it inhibitory or lethal. For example a “pro-barnase” can be constructed by linking a “pro” portion via, for example, the recognition sequence for tobacco etch virus NIa proteinase (Glu-X-X-Tyr-X-Gln Ser/Gly, where X=any amino acid) or tobacco vein mottling virus NIa (X-X-Val-Arg-/Lys-Phe/Thr-Gln Ser/Gly, where X=any amino acid).

In some embodiments, the multi-component system of the present invention is a 2-component system. The 2-component (two peptide) system, in which the 2-components are derived from a 1-component (single protein) can generally be derived from any single protein that has a cell-lethal or inhibitory function (depending only upon the protein folding constraints of the initial protein). Typically, the two peptides are from non-overlapping or minimally overlapping (e.g., 50, 35, 20, 15, 10, 5 or less) subsequences from a single inhibitory or cytotoxic protein. The peptides produced reassociate in the target cell reconstituting the function of the single peptide from which the 2 partial peptides are derived.

The secondary and tertiary structure of a host of proteins and the processes of protein folding are known to those of skill and provide the basis for designing 2-component peptide systems from a single protein. The 2 peptides will relate to the starting protein as 1) unmodified peptides that comprise the entire original protein, with the addition of a methionine or the conservative replacement of an amino acid with a methionine at the point of separation of the 2 peptides; 2) modified peptides as in (1) with the additional replacement of some amino acids by other amino acids designed to enhance the stability of the peptides and reassociated peptide complex; 3) modified peptides that comprise less than the full protein in toto; 4) peptides that are derived from only a portion of the original protein, where the portion of the original protein encodes a suitable function.

The design of non-functional polynucleotides or their encoded polypeptides can be achieved by a number of approaches well known to the skilled artisan. In the instant invention, these polynucleotides or polypeptides, when co-expressed in a cell, can confer lethality or some other desired function. These peptide subsequences, taken together, can be related to the original peptide as comprising the total protein sequence of the original functional protein, or as comprising a portion of the total protein sequence only. To ensure that sufficient temperature stability is retained in the now dimeric active protein, it may be necessary to incorporate specific amino acid changes into the partial coding sequences. The amino acid changes can be determined by examination of the original protein and the known amino acid interactions based on the protein structure as revealed through a range of physical techniques. In addition, the amino acid changes can be determined by random mutagenesis and screening of a combinatorial library of protein products. Alternatively, the amino acid changes can be determined by completely random mutagenesis and selection, using chemical treatments, PCR-induced mutagenesis, or other similar mutagenic treatments known to those skilled in the art.

The partial coding sequences derived from the original protein coding sequences is selected to retain activity of the reconstituted protein as well as a suitable level of stability with respect to environmental perturbations such as temperature changes. Several general routes can be taken to determining effective partial coding sequences.

Partial proteolysis

A number of proteins have been separated into distinct, resolvable domains through partial proteolysis. This is a rapid way to determine suitable coding sequences for the two non-functional polynucleotides or polypeptides of the instant invention. For example, pancreatic ribonuclease A can be cleared by subtilisin between residues 20 and 21, yielding a large and a small peptide, neither of which retains any activity, as essential catalytic residues are present in each peptide fragment. When the two peptides are mixed, the small peptide binds to the larger fragment and activity is reconstituted. In another example, staphylococcal nuclease can be resolved into three peptide fragments following partial proteolysis. Proteases initially cleave an intact protein at exposed residues, often ones that are part of exposed loops not involved in specific domains. Following partial proteolysis and analysis of the resulting peptide fragments by polyacrylamide gel electrophoresis to confirm that a simple digestion resulted, residual activity is evaluated. If activity is retained, the peptides are separated to determine whether neither peptide retains activity separately, and subsequently whether activity can be reconstituted upon remilling. Sequencing of the amino and carboxy termini of the two (or more) fragments reveals how to engineer the partial coding sequences in the instant invention.

Sequence conservation-based design

In situations where a number of sequences are available for proteins with the same function (e.g., subtilisin family proteins; colicin family proteins; ribonuclease family proteins), it is possible to identify regions that are not well conserved in all proteins. In combination with predictive analysis of secondary structure, it is possible to identify regions of the protein that are good candidates for separation into separate peptides. Such regions retain unaltered principal secondary structural features, such as alpha helices and beta-sheets. Within such regions, a number of possible replacement and coding sequence variants can be tested using an assay either for protein function in vitro, or for function, or for in vivo lethality.

Structure-based design

When a three-dimensional structure is available, from x-ray crystallography, NMR spectroscopy, etc., a more precise determination of candidate regions that would comprise the partial peptides of a single functional protein is possible. Analysis of the interactions between individual amino acids in a three-dimensional structure reveals subdomains of the original protein that have the potential to be separated and yet to bind to each other, and which sub-domains are likely to be non-functional when present separately. Additionally, analysis of these interactions reveals which amino acids, located between suitable sub-domains, are not involved in specific interactions with other amino acids in a way that would permit replacement with a methionine residue. Such an analysis is aided by additional sequence data for proteins with a high proportion of sequence relatedness to the starting protein. This provides additional evidence concerning residues that can be replaced with a methionine residue.

Exemplary polypeptides of the present invention include ribonucleases such as barnase (Mauguen et al., Nature, 297, 162-64, 1982), binase (Pavlovsky et al., FEBS Lett., 162, 167-70, 1983), Ribonuclease T1 (Fujii et al., Biosci. Biotechnol. Biochem. 59, 186 9-1874, 1995), nucleases such as colicin E9 (Wallis et al., Eur. J. Biochem, 220, 447-54, 1994) or BamHI (Newman et al., Science, 269, 656-63, 1995), and proteases such as subtilisin BPN′ (Eder et al., J. Mol. Biol., 233, 293-304) or other members of the subtilisin family. In some embodiments, these polypeptides are used to yield male sterility when co-expressed in tapetal tissue. Other polypeptides for creating cell toxicity or inhibition include those which produce toxic substances, disrupt cell function, lead to premature expression of glucanases, disrupt formation or secretion of substances required for pollen formation and disrupt mitochondrial function. In particularly preferred embodiments, the polypeptide is derived from a separate subsequence of a ribonuclease such as barnase. For barnase, the minimal length of each polypeptide is at least 20 amino acids. Generally, the extent of overlap of barnase polypeptides will be no more than 5 amino acids.

The enzyme barnase is a well-studied cell lethality function that has already been shown to be cell-autonomous, independent of other cellular functions, and very sensitive. Barnase expression has been shown to inhibit cell growth and development in specific plant tissues. The mature barnase protein consists of a 110 amino acid polypeptide. It has been shown in in vitro studies that amino acid 37 of the mature protein can be converted from a valine to a methionine with good retention of ribonuclease activity. Sancho and Fersht, J. Mol.Biol., 224, 741-47, (1992). It has also been shown that cyanogen bromide treatment cleaves the protein into a 36 amino acid peptide and a 74 amino acid peptide and that neither peptide retains any activity. Further, at least 30% of normal activity is reconstituted when the two peptides are mixed in vitro. Sancho and Fersht, (1992).

A particularly preferred embodiment is to produce the following two partial barnase coding subsequences via PCR amplification using a barnase gene as template and 1) primers designed to introduce a methionine codon at position 1 of the mature protein coding sequence and a stop codon after position 36 of the mature protein sequence, and 2) primers designed to introduce a methionine codon at position 37 of the mature protein coding sequence while leaving the end of the mature protein coding sequence intact. The two partial coding sequences can then be manipulated further to produce expression cassettes, using, for example, a promoter, a 5′ untranslated region, a 3′ untranslated region, and a polyadenylation signal. The two expression cassettes can be designed to create 2-component lethality systems that could be used to create a range of useful traits.

Means to assay for plant cell cytoxicity or inhibition produced by two peptide fragments of a single protein are well known in the art. For example, to determine whether the partial peptides designed as indicated above can be expressed separately without activity, but can be expressed together to give activity, enzymatic activity can be assayed directly on cell extracts containing the expressed peptides or in purified preparations of the peptides. Further, plant cell cytotoxicity or inhibition can be assayed using a range of indicators for cell function. In one preferred method, the expression cassettes can be introduced to cells along with an expression cassette that produces an easily assayed function, such as the beta-glucuronidase protein (Jefferson et al., EMBO J., 6, 3901-3907, 1987) or firefly luciferase (De Wet et al., Mol. Cell. Biol., 7, 725-37, 1987). If expression of the expression cassettes together is cytotoxic, then the amount of the reporter activity detected will be reduced compared with the activity detected when an expression cassette is introduced separately into a eukaryotic cell. Additionally, for example, two peptides derived from non-overlapping or minimally overlapping subsequences from a single inhibitory or cytotoxic protein such as a ribonuclease can be assayed for ribonucleolytic activity in vitro.

Transcripts

In addition to polypeptides, the transcription products of number of DNA constructs can be used to suppress expression of endogenous plant genes and yield a lethal result to the cell. These include cassettes which provide sense or antisense suppression, or ribozymes which, in combination with a second expression cassette, inhibit or kill the cell. Anti-sense RNA inhibition of gene expression has been shown; see, e.g., Sheehy et al., Proc. Nat. Acad. Sci. USA, 85:8805-8809 (1988), and Hiatt et al., U.S. Pat. No. 4,801,340. For examples of the use of sense suppression to modulate expression of endogenous genes see, Napoli et al., The Plant Cell 2:279-289 (1990), and U.S. Pat. No. 5,034,323.

Catalytic RNA molecules or ribozymes can also be used to inhibit gene expression. For example, in some embodiments a beneficial or lethal ribozyme can be transcribed upon induction by a polypeptide expressed from a second expression cassette (e.g., tet repressor/VP16 activator fusion polypeptide). It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. A general design and use of target RNA-specific ribozymes is described in Haseloff et al. Nature, 334:585-591 (1988).

For antisense suppression or sense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about 2000 nucleotides should be used, though a sequence of at least about 100 nucleotides is preferred, a sequence of at least about 200 nucleotides is more preferred, and a sequence of at least about 500 nucleotides is especially preferred.

Regeneration

Transformed plant cells which are derived by any number of transformation techniques can be cultured to regenerate a whole plant which possesses the transformed genotype and thus the desired expression cassette. Such regeneration techniques rely on manipulation of certain phytohormones in a tissue culture growth medium, typically relying on a biocide and/or herbicide marker which has been introduced together with the polynucleotide encoding a desired polypeptide. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

In some embodiments of the present invention, the expression cassettes encoding each component of the two or more component system are either introduced into a single cell by cotransformation of cells with each of the two expression cassettes, or by sequential transformation of cells with the two expression cassettes. When two promoters with overlapping specificity are used, cell inhibition or lethality will result in only the target tissue in which both promoters are sufficiently active.

In other embodiments the expression cassettes are introduced into different cells by transformation. Whole organisms are regenerated from the separated transformed cells, and then a hybrid organism is produced by crossing the individual organisms. In this way, the original whole organisms, each carrying a single expression cassette, show no cell inhibition or lethality. However, the hybrid organism resulting from the cross will have both expression cassettes in the same cell, and will express cell inhibitory function or lethality in a manner dependent upon the expression patterns of the chosen promoters.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

Target Cell Types

As the present invention can be used to eliminate particular cells or tissue types, a number of desired traits can thus be introduced into a plant. For example, to produce male sterility, expression is targeted to tapetal cells or pollen cells. To produce female sterility, each of the polynucleotide sequences is operably linked with a promoter expressed in stigmatic tissues, tissues of the transmitting tract, ovule tissues, or other tissues essential for female fertility. In order to produce seedless fruit, expression is targeted to embryos, endosperm, or other seed tissues.

Disease resistance in plants can be mediated by a hypersensitive response in which cells infected by a pathogen are killed to prevent further spread of the pathogen. Using promoters induced by pathogen attack and expression cassettes of the present invention, a synthetic hypersensitive response can be created. For example, tolerance to root knot or cyst nematodes can be mediated by eliminating the giant cells or specialized feeder cells these pests require for continued growth and multiplication in plant roots. Using promoters induced in the giant cells or specialized feeder cells in combination with expression cassettes of the present invention, these specialized root cells can be eliminated.

### EXAMPLE 1

Example 1 describes use of a repressor/activator fusion protein to induce expression of barnase in tapetal cells.

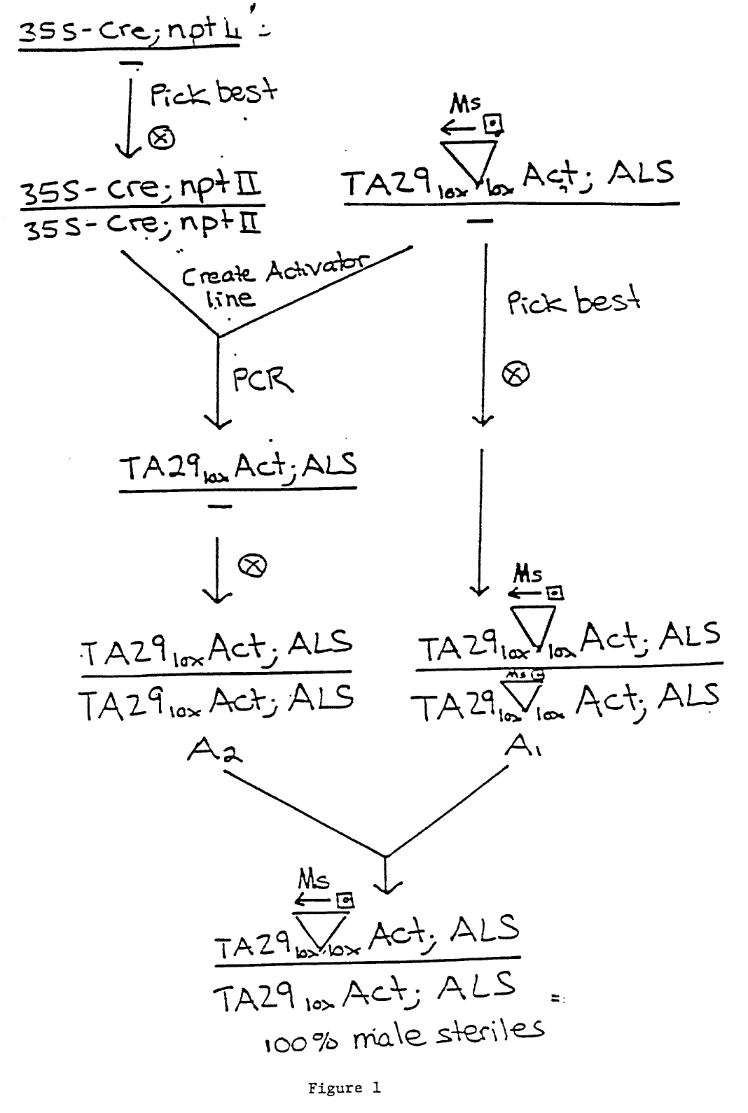

The method is diagramed in FIG. 1. In the Maintainer Field sub-lines A₁ and A₂, which are male fertile, are crossed to yield line A which is male sterile. Subline A₁ has a dominant male sterile gene (Ms) with an artificial promoter consisting of one or more tet operators and a TATA-box. In this condition, the male sterile gene is not transcribed and subline A₁ is male fertile. Subline A₂ has a tapetal-specific promoter driving the expression of a chimeric transcriptional activator. This transcriptional activator is made by fusing the tet repressor, which recognizes the tet operator, to a eukaryotic activation domain, the virion protein 16 (VP16) activation domain from Herpes simplex virus. This tet repressor/VP16 activator fusion (which is abbreviated in the diagram as “Act”) has been shown by Weinmann et al. (1994), supra, to activate transcription in plants from a minimal promoter plus 7 tet operators. Sublines A₁ and A₂ are crossed to produce line A, which is male sterile since it contains both the transcriptional activator (Act), which is expressed specifically in the tapetum, and the tet operator-Ms gene. The transcriptional activator binds to the tet operator inducing expression of the male sterile gene specifically in the tapetum. Tapetal-specific expression of Ms genes such as ribonucleases (e.g., Barnase), has been shown to produce male sterility. In the hybrid seed production field, line A, which is male sterile, can then be crossed to any line B, to produce hybrid seeds.

In many crops it is advantageous for the hybrids produced to be male fertile. This is also illustrated in FIG. 1, which shows the use of the cre/lox system to create two alternative alleles at one locus, one allele (A₁) consisting of the tet operator-Ms gene and the other allele (A₂) consisting of the tet repressor/VP16 activator driven off a tapetal-specific promoter. The initial transformant has a tet operator-Ms gene inserted in opposite orientation between a tapetal-specific promoter and the tet repressor/VP16 activator. Lox sites are placed in the same orientation on both sides of the tet operator-Ms insert. When made homozygous, this is used as line A₁. The tet repressor/activator in this line is silent since an insert is present in between the tapetal-specific promoter and the tet repressor/activator. The Ms gene is also silent, but is activated upon crossing to a line carrying a tet repressor/activator. Line A₂ is created by crossing line A₁ to a line carrying cre recombinase (from bacteriophage P1). Cre recombinase excises the tet operator-Ms gene, allowing the tet repressor/activator to be expressed in the tapetum. When made homozygous, this is used as line A₂.

### EXAMPLE 2

This example describes use of the AVR9 elicitor polypeptide from Cladosporium fulvum and the corresponding resistance gene, Cf9 from Lycopersicon esculentum to specifically kill tapetal cells.

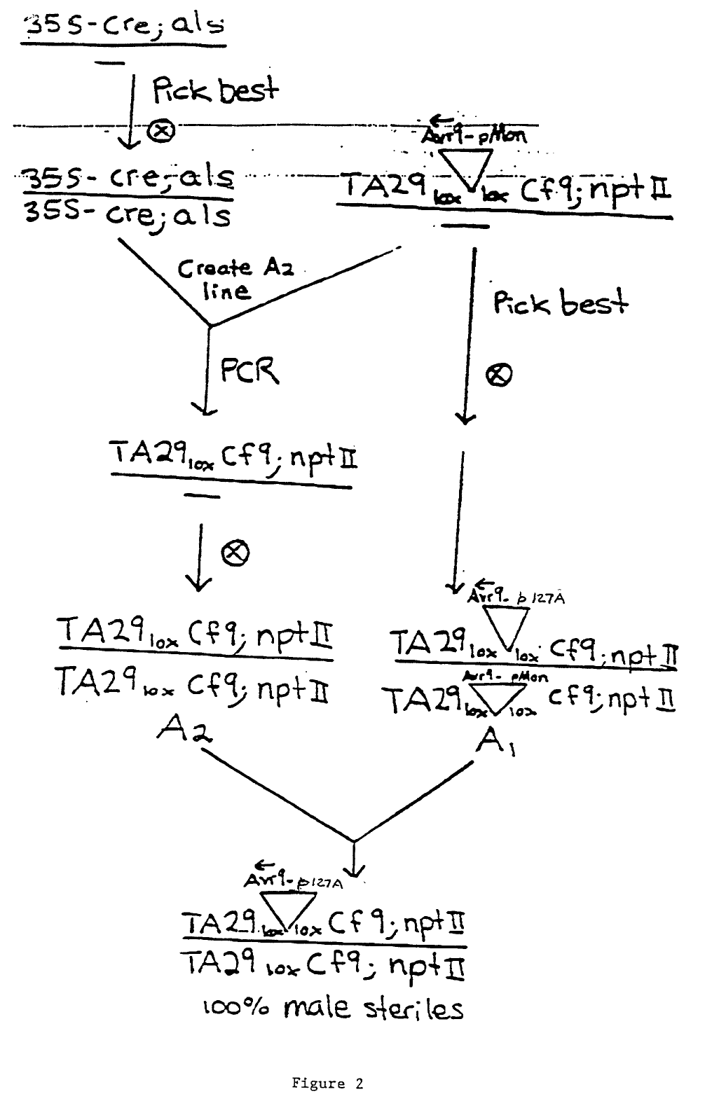

A method to produce hybrid seed using this embodiment is diagramed in FIG. 2. In the Maintainer Field sub-lines A₁ and A₂, which are male fertile, are crossed to yield line A which is male sterile. In subline A₁ the Cladosponum fulvum Avr9 avirulence gene is expressed off of a tapetal-specific promoter (p127a, described in U.S. Pat. No. 5,254,801). The AVR9 polypeptide is fused to a signal peptide from the tobacco Pr1a protein, as described in Hammond-Kosack et al. (1994). In subline A₂ the tomato Cf9 gene (the corresponding tomato resistance gene) is expressed off of a second tapetal-specific promoter (“TA29” in this illustration). Both sublines A₁ and A₂ are male fertile since the AVR9 and CF9 polypeptides, when expressed separately, do not confer cell-death. However, when sublines A₁ and A₂ are crossed together to produce line A, a hypersensitive response (HR) is initiated in the tapetum resulting in cell death. Hammond-Kosack et al. (1994) and Jones at al. (1994) have shown that cells expressing both AVR9 and CF9 become necrotic and that Cf9 expression is cell-autonomous. Tapetal cell-death should therefore confer male-sterility without adversely affecting other organs. The use of two distinct tapetal-specific promoters to express Cf9 and Avr9 greatly minimizes the risk that some expression might occur outside of the tapetum resulting in the death of that tissue. In the hybrid seed production field, line A, which is male sterile, can then be crossed to any line B, to produce hybrid seeds.

As in the previous example, the cre/lox system is used to create two alternative alleles at one locus, one allele (A₁) carrying the p127a-Avr9 gene and the other allele (A₂) carrying the TA29-Cf9 gene. The initial transformant has a p127a-Avr9 gene inserted in opposite orientation between a tapetal-specific promoter and Cf9. Lox sites are placed in the same orientation on both sides of the p127a-Avr9 insert. When made homozygous, this is used as line A₁. The TA29-Cf9 gene in this line is silent since an insert is present between the tapetal-specific promoter and the Cf9 gene. Only the p127a-Avr9 gene will be expressed in this line. Line A₂ is created by crossing line A₁ to a line carrying cre recombinase (from bacteriophage P1). Cre recombinase would excise the p127a-Avr9 gene, allowing the TA29-Cf9 gene to be expressed. When made homozygous, this is used as line A₂.

### EXAMPLE 3

This example describes use of the cre-lox system to insert two functional expression cassettes into a lox site previously introduced into a plant genome. The method uses a combination of mutant lox sites as described by Albert et al. Plant J. 7:649-659 (1995) to increase efficiency of the insertion event as compared to excision.

A mutant lox site (lox₆₆, as described by Albert et al.) is introduced into a desired plant genome using a recombinant expression cassette having a CaMV35S promoter linked to a structural gene encoding cre. The lox₆₆ site is placed between the structural gene and the promoter. Protoplasts from this plant are then transformed with a plasmid carrying a second mutant site (lox₇₁) linked to a selectable marker such as hygromycin phosphotransferase (hpt) plus a first functional expression cassette (e.g., AVR9 under control of a tapetal-specific promoter). Insertion of the hpt at the lox site yields a 35S-lox_{wt}-hpt, which provides a selectable marker (hygromycin resistance). The wild-type lox site is reconstructed from the cross-over event between the two mutant sites. Farther downstream, a second double mutant lox site is created, which prevents cross-over events at the insertion sites, thereby preventing excision of the inserted fragment. In addition, insertion renders the 35S-cre expression cassette non-functional, thereby preventing continued expression of the cre recombinase, which interferes with stable integration.

The above process is repeated using the original plant line and a second plasmid carrying a second functional expression cassette (e.g., CF9 under control of a tapetal-specific promoter). After appropriate selfing, two regenerated plant lines, one homozygous for the first expression cassette and one homozygous for the second expression cassette are created. A cross between these two lines leads to heterozygous plants in which the two proteins are specifically expressed in tapetal cells.

An alternate means for using insertion to create alternate alleles is to transform one plant with the 35S-lox₆₆-cre construct described above and a second plant with a construct comprising the following elements: lox₇₁-hpt-AVR9 under control of a tapetal-specific promoter-lox₇₁. When the two plants are crossed the hpt-AVR9 sequence will be excised from its site of insertion and will integrate at the lox₆₆ site between the 35S promoter and the cre structural gene. Selection for hygromycin resistance allows selection of transformed cells.

As in the first method, the procedure is repeated with a second expression cassette, CF9 under control of a tapetal-specific promoter. Appropriate selfing and crosses leads to heterozygous plants in which the two proteins are specifically expressed in tapetal cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A plant containing a plant cell comprising a first and a second expression cassette located at the same locus on each of two homologous chromosomes, wherein:
the first expression cassette present on a first chromosome homolog comprises a first plant promoter operably linked to a first polynucleotide sequence encoding a first polypeptide, wherein a recombinase site is present between the first promoter and the first polynucleotide sequence;
the second expression cassette present on a second chromosome homolog comprises the first plant promoter inoperably linked to the first polynucleotide sequence, wherein an intervening expression cassette is flanked by two recombinase sites and situated between the first promoter and the first polynucleotide sequence of the second expression cassette, the intervening expression cassette comprising a second plant promoter operably linked to a second polynucleotide sequence encoding a second polypeptide; and
wherein the presence of the first and second polypeptides in a cell is lethal to the cell.

2. The plant of claim 1, wherein the recombinase sites are lox sites.

3. The plant of claim 1, wherein the first polypeptide is a transactivator protein.

4. The plant of claim 1, wherein the intervening expression cassette is in reverse orientation with respect to the second expression cassette.

5. The plant of claim 3, wherein the second polypeptide is lethal to plant cells.

6. The plant of claim 5, wherein the second polypeptide is a ribonuclease.

7. The plant of claim 6, wherein the ribonuclease is Barnase.

8. The plant of claim 1, wherein the first polypeptide is an avirulence gene product derived from a plant pathogen and the second polypeptide is a resistance gene product associated with the avirulence gene.

9. The plant of claim 8, wherein the first polypeptide is AVR9.

10. The plant of claim 9, wherein the second polypeptide is CF9.

11. The plant of claim 1, wherein the first or the second promoter is a tissue-specific promoter.

12. The plant of claim 1, wherein the first and second promoters are each functional in tapetal cells.

13. The plant of claim 1, wherein the first and second polypeptides each comprise a separate subsequence of a single functional polypeptide.

14. A method of modifying cellular function in a plant, the method comprising the steps of:
introducing into a plant a first expression cassette comprising a first plant promoter operably linked to a first polynucleotide encoding a first polypeptide, wherein a recombinase site is present between the first promoter and the first polynucleotide;
introducing into the plant a second expression cassette comprising the first plant promoter inoperably linked to a polynucleotide encoding the first polypeptide, wherein an intervening expression cassette is flanked by recombinase sites and situated between the first promoter and the first polypeptide of the second expression cassette, the intervening expression cassette comprising a plant promoter operably linked to a polynucleotide encoding a second polypeptide; and
wherein the presence of the first and second polypeptides in a cell is lethal to the cell.

15. The method of claim 14, wherein the two expression cassettes are introduced through a sexual cross and the two expression cassettes are present on chromosome homologs.

16. The method of claim 14, wherein the recombinase sites are lox sites.

17. The method of claim 14, wherein the first polypeptide is a transactivator protein.

18. The method claim 14, wherein the intervening expression cassette is in reverse orientation with respect to the second expression cassette.

19. The method of claim 17, wherein the second polypeptide is lethal to plant cells.

20. The method of claim 19, wherein the second polypeptide is a ribonuclease.

21. The method of claim 20, wherein the ribonuclease is Barnase.

22. The method of claim 14, wherein the first polypeptide is an avirulence gene product derived from a plant pathogen and the second polypeptide is a resistance gene product associated with the avirulence gene.

23. The method of claim 22, wherein the first polypeptide is AVR9.

24. The method of claim 23, wherein the second polypeptide is CF9.

25. The method of claim 14, wherein the first or the second promoter is a tissue-specific promoter.

26. The method of claim 14, wherein the first and second promoters are each functional in tapetal cells.

27. The method of claim 14, wherein the first and second polypeptides each comprise a separate subsequence of a single functional polypeptide.
